(19) United States
(12) Patent Application Publication
(10) Pub. No.: US 2025/0079013 A1
(43) Pub. Date: Mar. 6, 2025

(54) NOTIFICATION DEVICE, NOTIFICATION METHOD AND PROGRAM

(71) Applicant: Nippon Telegraph and Telephone Corporation, Tokyo (JP)

(72) Inventors: Yuki Hashimoto, Tokyo (JP); Kazuhiko Takagahara, Tokyo (JP)

(21) Appl. No.: 18/546,371

(22) PCT Filed: Feb. 26, 2021
(86) PCT No.: PCT/JP2021/007285

§ 371 Date: Aug. 14, 2023

(51) Int. Cl. G16H 50/30; G16H 10/60; G16H 40/67

(52) U.S. Cl. CPC G16H 50/30; G16H 10/60; G16H 40/67

(57) ABSTRACT

A notification device includes a calculation parameter acquisition unit that acquires information regarding physical features, work conditions, and environmental conditions of a measurement target person, a biological information prediction unit that predicts biological information of the measurement target person for each of times in a case where the measurement target person performs work under conditions specified by the work conditions and the environmental conditions on the basis of the information acquired by the calculation parameter acquisition unit, a time prediction unit that predicts a time at which the biological information predicted by the biological information prediction unit will exceed a predetermined threshold value, and a heat risk notification unit that notifies a work manager or the measurement target person of heat countermeasure information based on the time predicted by the time prediction unit.

### CROSS-REFERENCE TO RELATED APPLICATIONS

This application is a national phase entry of PCT Application No. PCT/JP2021/007285, filed on Feb. 26, 2021, which application is hereby incorporated herein by reference.

### TECHNICAL FIELD

The present invention relates to a notification device, a notification method, and a program for providing information useful for preventing heat stroke of a measurement target person.

### BACKGROUND

The number of deaths and injuries due to heat stroke in workplaces in 2019 was 790, which is the second highest after the number of deaths and injuries of 1178 in 2018 when comparing the occurrence situation in the past 10 years (2010 to 2019). By industry type, the number of people in the construction industry was 147, and the number of people in the manufacturing industry was 172, and it can be seen that the number of occurrences of heat stroke is large in industries in which it is likely there will be direct exposure to sunlight or a high-temperature and high-humidity environment (refer to Non Patent Literature 1).

In such a work site, self-management of a worker, time management for the worker by a work manager, vocal physical condition management of the worker by the work manager, and the like may be performed. However, in work management that depends only on subjective information such as self-reporting, there is a possibility that a change in physical condition caused by a load or the like that the worker receives from a hot environment will be overlooked.

As a physical condition management method that does not depend on subjective information, there is a case where a method is employed in which a work manager gives a message for replenishing moisture and salt, a message for encouraging a break, and the like on the basis of a numerical value of a heat index such as wet bulb globe temperature (WBGT). However, the WBGT is a numerical value calculated on the basis of an outdoor observation point in a specific region, and is a reference value that does not reflect an environmental change according to a behavior of an individual worker. Therefore, in the physical condition management method based on the WBGT, it can be said that there is a possibility of overlooking a change in a physical condition of a worker similarly to the above-described subjective information.

On the other hand, as a physical condition management method focusing on personal biological information, there is a method of monitoring a deep body temperature of a worker. For example, in the American Conference of Governmental Industrial Hygienists (AGCIH), when the deep body temperature is 38.5° C. or higher in the case of a heat acclimated person and 38° C. or higher in the case of a heat unacclimated person, this is used as a reference for work stoppage due to summer heat (refer to Non Patent Literature 2).

An index focusing on biological information is useful in monitoring heat stroke onset risk for each individual. However, in order to measure biological information, there is a problem that a measuring device such as a wearable device needs to be worn for each individual.

### CITATION LIST

### Non Patent Literature

Non Patent Literature 1: “Circumstances of occurrence of fatalities and injuries caused by heatstroke in the workplace in 2019”, the Ministry of Health, Labour and Welfare, [Retrieved Oct. 30, 2020], <https://www.mhlw.go.jp/content/11303000/000612135.pdf>.
Non Patent Literature 2: “Study on heat stroke countermeasures in the workplace (part 2)”, National Institute of Occupational Safety and Health, National Institute of Health, [Retrieved Oct. 30, 2020],
   <https://www.jniosh.johas.go.jp/publication/mail_mag/2013/61-column.html>.

### SUMMARY

### Technical Problem

Embodiments of the present invention have been made to solve the above problems, and an object of embodiments of the present invention is to provide information useful for prevention of heat stroke of a measurement target person without mounting a measuring device such as a wearable device on the measurement target person.

### Solution to Problem

According to embodiments of the present invention, there is provided a notification device including a calculation parameter acquisition unit configured to acquire information regarding physical features, work conditions, and environmental conditions of a measurement target person; a biological information prediction unit configured to predict biological information of the measurement target person for each of times in a case where the measurement target person performs work under conditions specified by the work conditions and the environmental conditions on the basis of the information acquired by the calculation parameter acquisition unit; a time prediction unit configured to predict a time at which the biological information predicted by the biological information prediction unit will exceed a predetermined threshold value; and a heat risk notification unit configured to notify a work manager or the measurement target person of heat countermeasure information based on the time predicted by the time prediction unit.

In a configuration example of the notification device of embodiments of the present invention, the information regarding the physical features includes information regarding a height, a weight, an age, and a sex of the measurement target person, the information regarding the work conditions includes information regarding clothing and a metabolic equivalents (METs) value of the measurement target person, and the information regarding the environmental conditions includes information regarding a temperature and a humidity on the day of work at a place where the measurement target person is performing work.

In a configuration example of the notification device of embodiments of the present invention, the biological information is a deep body temperature of the measurement target person, the time prediction unit predicts a time at which the deep body temperature predicted by the biological information prediction unit will exceed a predetermined deep body temperature threshold value, and the heat countermeasure information includes information for reporting the time predicted by the time prediction unit as a time at which the measurement target person will be exposed to an excessive heat load.

In a configuration example of the notification device of embodiments of the present invention, the biological information is a water loss amount of the measurement target person, the time prediction unit predicts a time at which the water loss amount predicted by the biological information prediction unit will exceed a predetermined water loss amount threshold value, and the heat countermeasure information includes information for reporting the time predicted by the time prediction unit as a time at which the measurement target person will be exposed to an excessive heat load.

In a configuration example of the notification device of embodiments of the present invention, the biological information is a deep body temperature and a water loss amount of the measurement target person, the time prediction unit predicts a time at which the deep body temperature predicted by the biological information prediction unit will exceed a predetermined deep body temperature threshold value and a time at which the water loss amount predicted by the biological information prediction unit will exceed a predetermined water loss amount threshold value, and the heat countermeasure information includes information for reporting an earlier one of the two times predicted by the time prediction unit as a time at which the measurement target person will be exposed to an excessive heat load.

In a configuration example of the notification device of embodiments of the present invention, the heat countermeasure information includes the biological information predicted by the biological information prediction unit in addition to the information regarding the time at which the measurement target person will be exposed to the excessive heat load.

According to embodiments of the present invention, there is provided a notification method including a first step of acquiring information regarding physical features, work conditions, and environmental conditions of a measurement target person; a second step of predicting biological information of the measurement target person for each of times in a case where the measurement target person performs work under conditions specified by the work conditions and the environmental conditions on the basis of the information acquired in the first step; a third step of predicting a time at which the biological information predicted in the second step will exceed a predetermined threshold value; and a fourth step of notifying a work manager or the measurement target person of heat countermeasure information based on the time predicted in the third step.

According to embodiments of the present invention, there is provided a program causing a computer to execute each of the above steps.

### Advantageous Effects of Embodiments of the Invention

According to embodiments of the present invention, by providing the calculation parameter acquisition unit, the biological information prediction unit, the time prediction unit, and the heat risk notification unit, it is possible to predict the biological information of the measurement target person after the start of work before the start of work, predict the time at which the measurement target person will be exposed to an excessive heat load, and notify the work manager or the measurement target person of the heat countermeasure information based on the prediction result, without mounting a measuring device such as a wearable device on the measurement target person. Therefore, according to the present embodiment, it is possible to implement a measure useful for preventing heat stroke of the measurement target person.

### BRIEF DESCRIPTION OF THE DRAWINGS

FIG. 1 is a block diagram illustrating a configuration of a notification device according to a first embodiment of the present invention.

FIG. 2 is a flowchart for describing an operation of the notification device according to the first embodiment of the present invention.

FIG. 3 is a block diagram illustrating a configuration of a biological information prediction unit of the notification device according to the first embodiment of the present invention.

FIG. 4 is a block diagram illustrating a configuration of a deep body temperature prediction unit according to a second embodiment of the present invention.

FIG. 5 is a block diagram illustrating a configuration of a deep body temperature prediction unit according to a third embodiment of the present invention.

FIG. 6 is a diagram illustrating a two-site two-layer model of the body of a measurement target person and a heat quantity flowing into and out of each site/each layer of the measurement target person.

FIG. 7 is a flowchart for describing an operation of the deep body temperature prediction unit according to the third embodiment of the present invention during main calculation.

FIG. 8 is a flowchart for describing an operation of the deep body temperature prediction unit according to the third embodiment of the present invention during preliminary calculation I.

FIG. 9 is a flowchart for describing an operation of a deep body temperature prediction unit according to the third embodiment of the present invention during preliminary calculation II.

FIG. 10 is a flowchart for describing an operation of the deep body temperature prediction unit according to the third embodiment of the present invention during preliminary calculation III.

FIG. 11 is a flowchart for describing an operation of the deep body temperature prediction unit according to the third embodiment of the present invention during main calculation.

FIG. 12 is a block diagram illustrating a configuration example of a computer that realizes the notification device according to the first to third embodiments of the present invention.

### DETAILED DESCRIPTION OF ILLUSTRATIVE EMBODIMENTS

### First Embodiment

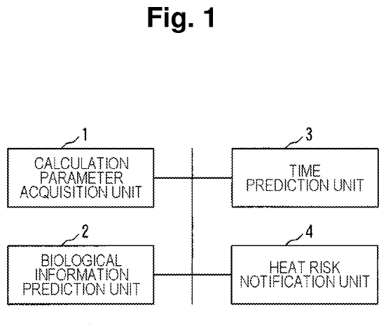

Hereinafter, embodiments of the present invention will be described with reference to the drawings. FIG. 1 is a block diagram illustrating a configuration of a notification device according to a first embodiment of the present invention. The notification device of the present embodiment includes a calculation parameter acquisition unit 1, a biological information prediction unit 2, a time prediction unit 3, and a heat risk notification unit 4.

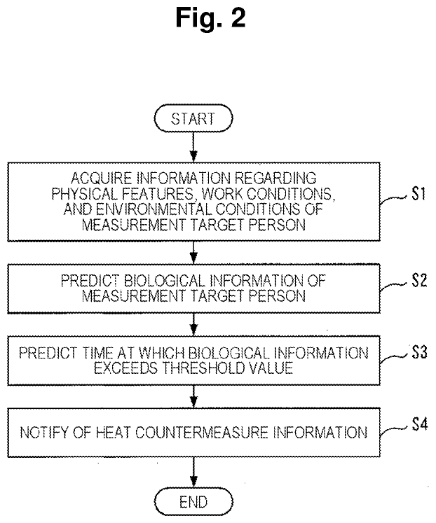

FIG. 2 is a flowchart for describing an operation of the notification device of the present embodiment. The calculation parameter acquisition unit 1 of the notification device acquires in advance information regarding physical features of a measurement target person, information regarding work conditions of the measurement target person, and information regarding environmental conditions of the measurement target person, which are necessary for prediction of biological information (step S1 in FIG. 2).

Examples of the information regarding the physical features of the measurement target person include height, weight, sex, age, and information regarding the presence or absence of heat acclimation of the measurement target person. Examples of the information regarding the work conditions of the measurement target person include information regarding clothing of the measurement target person and exercise load information (metabolic equivalents (METs)). Examples of the information regarding the environmental conditions of the measurement target person include temperature, humidity, wind speed, and evaporation heat of water at a work site on the day of work.

The above information necessary for the prediction of the biological information may be input by a work manager or an operator of the notification device. The calculation parameter acquisition unit 1 may acquire the information regarding the physical features of the measurement target person from a terminal such as a smartphone of the measurement target person connected to the notification device of the present embodiment via a network. When a dedicated application is installed in advance in the terminal owned by the measurement target person, the information regarding the physical features of the measurement target person can be acquired via the application.

Similarly, the calculation parameter acquisition unit 1 may acquire information regarding the work conditions of the measurement target person from the terminal of the measurement target person. In this case, the measurement target person may select clothes to be worn on the day of work or work details from among options by operating the terminal owned by the measurement target person. The details selected by the measurement target person is transmitted to the notification device. The calculation parameter acquisition unit 1 may select information regarding work conditions corresponding to the details reported from the measurement target person from information prepared in advance.

The information regarding the environmental conditions of the measurement target person may be actually measured by a measurement unit provided in the biological information prediction unit 2 as will be described later, or a predicted value of temperature, a predicted value of humidity, and a predicted value of wind speed may be acquired from an external weather prediction system.

Next, on the basis of the information acquired by the calculation parameter acquisition unit 1, the biological information prediction unit 2 of the notification device predicts biological information of the measurement target person at each time in a case where the measurement target person performs work under conditions specified by the work conditions and the environmental conditions (step S2 in FIG. 2).

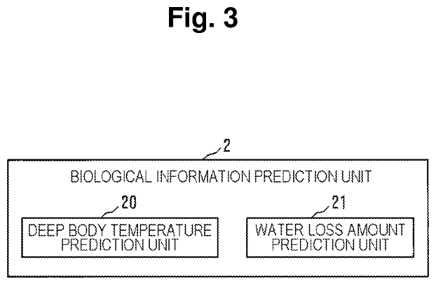

FIG. 3 is a block diagram illustrating a configuration of the biological information prediction unit 2. The biological information prediction unit 2 includes a deep body temperature prediction unit 20 and a water loss amount prediction unit 21.

The deep body temperature prediction unit 20 predicts a deep body temperature of the measurement target person at each future time in a case where the measurement target person performs work under conditions specified by the work conditions and the environmental conditions on the basis of the information regarding the physical features, the work conditions, and the environmental conditions of the measurement target person stored in advance in the calculation parameter acquisition unit 1. A specific example of the deep body temperature prediction unit 20 will be described later.

The water loss amount prediction unit 21 predicts a water loss amount at each future time of the measurement target person in a case where the work is performed under the conditions specified by the work conditions and the environmental conditions on the basis of the information regarding the physical features, the work conditions, and the environmental conditions of the measurement target person stored in advance in the calculation parameter acquisition unit 1. A specific example of the water loss amount prediction unit 21 will be described later.

The biological information prediction unit 2 may predict only a deep body temperature of the measurement target person with the deep body temperature prediction unit 20, may predict only a water loss amount of the measurement target person with the water loss amount prediction unit 21, or may predict both the deep body temperature and the water loss amount.

Next, the time prediction unit 3 predicts a time at which the biological information predicted by the biological information prediction unit 2 will exceed a predetermined threshold value (step S3 in FIG. 2).

A deep body temperature threshold value is stored in advance in the calculation parameter acquisition unit 1. A predicted value of the deep body temperature of the measurement target person at each future time can be obtained by the deep body temperature prediction unit 20 of the biological information prediction unit 2. Therefore, the time prediction unit 3 can predict a time at which the deep body temperature of the measurement target person will exceed the deep body temperature threshold value.

The calculation parameter acquisition unit 1 may store two types of the deep body temperature threshold value of a heat unacclimated person and the deep body temperature threshold value of a heat acclimated person. According to Non Patent Literature 2, the deep body temperature threshold value of a heat unacclimated person is 38° C., and the deep body temperature threshold value of a heat acclimated person is 38.5° C. In a case where the information regarding the presence or absence of heat acclimation is stored in the calculation parameter acquisition unit 1 as the information regarding the physical features of the measurement target person, the time prediction unit 3 may employ a corresponding deep body temperature threshold value according to the presence or absence of heat acclimation of the measurement target person.

A water loss amount threshold value (limit perspiration rate) is stored in advance in the calculation parameter acquisition unit 1. A predicted value of the water loss amount of the measurement target person at each future time can be obtained by the water loss amount prediction unit 21 of the biological information prediction unit 2. Therefore, the time prediction unit 3 can predict a time at which an integrated value of the water loss amount of the measurement target person will exceed the water loss amount threshold value.

As in the case of the deep body temperature, the calculation parameter acquisition unit 1 may store two types of water loss amount threshold values of a heat unacclimated person and a heat acclimated person. According to Non Patent Literature 2, the water loss amount threshold value of a heat unacclimated person is 1 L/h, and the water loss amount threshold value of a heat acclimated person is 1.25 L/h. In a case where the information regarding the presence or absence of heat acclimation is stored in the calculation parameter acquisition unit 1 as the information regarding the physical features of the measurement target person, the time prediction unit 3 may employ a corresponding water loss amount threshold value according to the presence or absence of heat acclimation of the measurement target person.

The time prediction unit 3 may calculate a percentage obtained by dividing a value obtained by converting an integrated value of the water loss amount into a weight integrated value by the weight of the measurement target person, and predict a time at which a calculated value will exceed the water loss amount threshold value per weight. According to Non Patent Literature 2, the water loss amount threshold value (body weight loss rate) per body weight is 1.5%.

Next, the heat risk notification unit 4 notifies the work manager or the measurement target person of heat countermeasure information based on the time predicted by the time prediction unit 3 (step S4 in FIG. 2). The heat countermeasure information includes information for reporting the time predicted by the time prediction unit 3 as a time at which the measurement target person will be exposed to an excessive heat load.

As described above, in a case where the deep body temperature and the water loss amount of the measurement target person are predicted by the biological information prediction unit 2, the time prediction unit 3 predicts two times, such as the time at which the deep body temperature of the measurement target person will exceed the deep body temperature threshold value and the time at which the integrated value of the water loss amount of the measurement target person will exceed the water loss amount threshold value. In this case, the heat risk notification unit 4 may notify the work manager or the measurement target person of an earlier one of the two times predicted by the time prediction unit 3 as a time at which the measurement target person will be exposed to an excessive heat load.

In a case where a time prediction result cannot be obtained from the time prediction unit 3, that is, in a case where it is estimated that there is no time at which the biological information of the measurement target person will exceed the threshold value, the heat risk notification unit 4 may notify the work manager or the measurement target person that there is no time at which the measurement target person will be exposed to an excessive heat load.

Examples of timings of prediction and notification include a timing before the start of work on the day of work of the measurement target person. In addition to the above time, the heat countermeasure information to be reported may include biological information such as a deep body temperature and a water loss amount, a message indicating that the measurement target person will be exposed to an excessive heat load, a message prompting the measurement target person to take a break, and a message for prompting the measurement target person to rehydrate or retreat to a cool place.

The heat risk notification unit 4 may transmit the heat countermeasure information to a terminal such as a smartphone owned by the work manager or a smartphone owned by the measurement target person via a wired network or a wireless network.

As described above, in the present embodiment, it is possible to predict biological information of the measurement target person after the start of work before the start of work, predict a time at which the measurement target person will be exposed to an excessive heat load, and notify the work manager or the measurement target person of the heat countermeasure information based on the predicted result without wearing a measuring device such as a wearable device on the measurement target person. Therefore, according to the present embodiment, it is possible to implement a measure useful for preventing heat stroke of the measurement target person who is working.

### Second Embodiment

Next, a second embodiment of the present invention will be described. The present embodiment describes a specific example of the deep body temperature prediction unit 20 of the first embodiment, and is based on the configuration disclosed in JP 2020-065823 A.

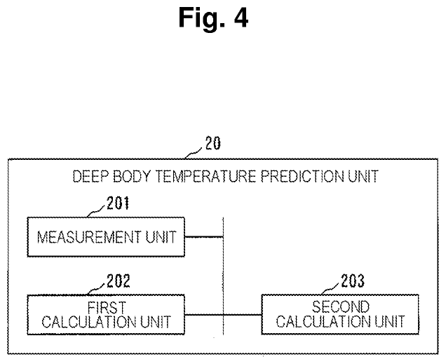

FIG. 4 is a block diagram illustrating a configuration of the deep body temperature prediction unit 20 of the present embodiment. The deep body temperature prediction unit 20 of the present embodiment includes a measurement unit 201, a first calculation unit 202, and a second calculation unit 203.

The measurement unit 201 measures a temperature in the vicinity of the measurement target person (the temperature of the atmosphere of the measurement target person). The first calculation unit 202 obtains a heat quantity Q[W] generated due to work of the measurement target person on the basis of information stored in advance in the calculation parameter acquisition unit 1. Specifically, the first calculation unit 202 acquires the METs of the measurement target person stored in advance in the calculation parameter acquisition unit 1, and calculates the heat quantity Q[W] as follows.

$$Equation 1$$

$$Q = METS \times 1.05 \times weight \times 4184 \div 3600 \qquad ( 1 )$$

Weight is a weight [kg] of the measurement target person, and is stored in the calculation parameter acquisition unit 1.

Next, the second calculation unit 203 predicts a deep body temperature of the measurement target person from the heat quantity Q[W] calculated by the first calculation unit 202, a heat exchange amount between the atmosphere of the measurement target person and the measurement target person obtained from the air temperature measured by the measurement unit 201, and an evaporation amount of sweat obtained from a perspiration amount of the measurement target person.

Hereinafter, the prediction of the deep body temperature in the second calculation unit 203 will be described in more detail. The second calculation unit 203 predicts a deep body temperature T [° C.] of the measurement target person at the future time t according to the following Equation 2.

$$Equation 2$$

$$T = {T}_{- 1} + \frac{[ Q ( t ) - {\int }_{S} H \times \{ ( {T}_{- 1} - {T}_{0} ) + ( 30.0 - {T}_{a} ) \} ds - ( {T}_{- 1} - {T}_{0} ) \times sw ( t ) ]}{weight \times C} Δ t \qquad ( 2 )$$

Q(t)[W] is a heat quantity calculated by the first calculation unit 202. Tₐ[° C.] is the air temperature measured by the measurement unit 201. sw(t)[W] is a heat quantity derived by evaporation of perspiration of the measurement target person at time t. T₀[° C.] is an initial value of the deep body temperature of the measurement target person. S [m²] is a body surface area of the measurement target person. H [W/(m²·° C.)] is a heat transfer coefficient between the skin of the measurement target person and the atmosphere. C [J/(kg·° C.)] is specific heat of the measurement target person. T₋₁[° C.] is a deep body temperature of the measurement target person at a time before time t by Δt [s].

In Equation 2, “∫ₛH×{(T₋₁−T₀)+(30.0−Tₐ)}dS” represents heat exchange with the atmosphere. The second term on the right side of Equation 2 corresponds to an increasing value of the deep body temperature of the measurement target person as Δt elapses. The initial value T₀[° C.] of the deep body temperature of the measurement target person is stored in advance in the calculation parameter acquisition unit 1 as a value actually measured in a state in which the measurement target person is at rest. The body surface area S [m²] of the measurement target person is as follows.

$$Equation 3$$

$$S = {height}^{0.725} \times {weight}^{0.425} \times 7.184 \times {10}^{- 3} \qquad ( 3 )$$

The calculation formula of the body surface area S in Equation 3 is known as Duboi's formula. Height is a height [cm] of the measurement target person, and is stored in advance in the calculation parameter acquisition unit 1. The heat transfer coefficient H [W/(m²·° C.)] between the skin of the measurement target person and the atmosphere and the specific heat C [J/(kg·° C.)] of the measurement target person are also stored in advance in the calculation parameter acquisition unit 1 as the information regarding the physical features.

The heat quantity sw(t)[W] deprived by evaporation of perspiration of the measurement target person at time t is as follows.

$$Equation 4$$

$$sw ( t ) = [ \text{} {α}_{11} \{ {β}_{11} ( {T}_{- 1} - {T}_{0} ) - {β}_{10} \} + {α}_{10} ] + [ \text{} {α}_{21} tan h \{ {β}_{21} ( {T}_{- 1} - {T}_{0} ) - {β}_{20} \} + {α}_{20} ] \qquad ( 4 )$$

In Equation 4, appropriate values are given to the constants α₁₀, α₁₁, α₂₀, α₂₁, β₁₀, β₁₁, β₂₀, and β₂₁. These constants are set depending on the ease of sweating of the measurement target person, and are stored in advance in the calculation parameter acquisition unit 1 as the information regarding the physical features of the measurement target person.

Thus, the deep body temperature prediction unit 20 of the present embodiment can predict the deep body temperature T [° C.] of the measurement target person at each future time.

In the present embodiment, a temperature in the vicinity of the measurement target person is measured by the measurement unit 201, but a predicted value of the air temperature Tₐ[° C.] of the measurement target person on the day of work may be acquired from the calculation parameter acquisition unit 1 and used.

### Third Embodiment

Next, a third embodiment of the present invention will be described. The present embodiment describes a specific example of the deep body temperature prediction unit 20 and the water loss amount prediction unit 21 of the first embodiment, and describes a configuration different from that of the second embodiment.

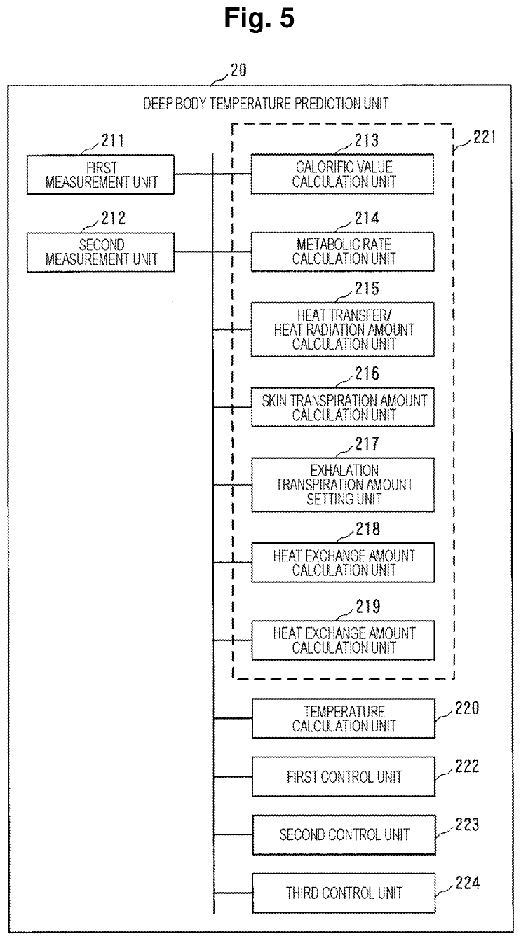

FIG. 5 is a block diagram illustrating a configuration of the deep body temperature prediction unit 20 of the present embodiment. The deep body temperature prediction unit 20 of the present embodiment includes a first measurement unit 211 that measures the temperature in the vicinity of the measurement target person, a second measurement unit 212 that measures the humidity in the vicinity of the measurement target person, a calorific value calculation unit 213 that calculates a calorific value of each of the trunk (first site) and the limbs (second site) due to motion of the measurement target person on the basis of a setting that the measurement target person is in a resting state during first and second preliminary calculations and calculates a calorific value on the basis of information in the calculation parameter acquisition unit 1 during main calculation, and a metabolic rate calculation unit 214 that calculates a metabolic rate of the trunk skin layer, a metabolic rate of the trunk deep layer, a metabolic rate of a limb skin layer, and a metabolic rate of a limb deep layer of the measurement target person on the basis of a skin temperature and a deep part temperature at the time immediately before calculated by the temperature calculation unit 220 during the first and second preliminary calculations and the main calculation.

The deep body temperature prediction unit 20 includes a heat transfer/heat radiation amount calculation unit 215 (first heat exchange amount calculation unit) that calculates a first heat exchange amount between the skin and the outside air in each of the trunk and the limbs of the measurement target person on the basis of a temperature corresponding to the temperature neutral range during the first preliminary calculation and the skin temperature at the time immediately before calculated by the temperature calculation unit 220, and calculates the first heat exchange amount on the basis of the information in the calculation parameter acquisition unit 1 and the skin temperature at the time immediately before calculated by the temperature calculation unit 220 during the second preliminary calculation and the main calculation, and a skin transpiration amount calculation unit 216 that calculates a skin transpiration amount in each of the trunk and the limbs of the measurement target person on the basis of a fixed value of the temperature corresponding to the temperature neutral range, a fixed value of the humidity, and the skin temperature at the time immediately before calculated by the temperature calculation unit 220 and the deep part temperature during the first preliminary calculation, and calculates the skin transpiration amount on the basis of the information in the calculation parameter acquisition unit 1, the skin temperature and the deep part temperature at the time immediately before calculated by the temperature calculation unit 220 during the second preliminary calculation and the main calculation.

The deep body temperature prediction unit 20 includes an exhalation transpiration amount setting unit 217 (fourth heat exchange amount setting unit) that sets a fourth heat exchange amount between the outside air and the deep part of the measurement target person, a heat exchange amount calculation unit 218 (second heat exchange amount calculation unit) that calculates a second heat exchange amount between the deep part and the skin in each of the trunk and the limbs of the measurement target person on the basis of the setting that the measurement target person is in the resting state and the skin temperature and the deep part temperature at the time immediately before calculated by the temperature calculation unit 220 during the first and second preliminary calculations, and calculates the second heat exchange amount on the basis of the information in the calculation parameter acquisition unit 1, the skin temperature and the deep part temperature at the time immediately before calculated by the temperature calculation unit 220 during the main calculation, a heat exchange amount calculation unit 219 (third heat exchange amount calculation unit) that calculates a third heat exchange amount between the trunk and the limbs in the deep part of the measurement target person on the basis of the skin temperature and the deep part temperature at the time immediately before calculated by the temperature calculation unit 220 during the first and second preliminary calculations and the main calculation, and the temperature calculation unit 220 that calculates the skin temperature and the deep part temperature of each of the trunk and the limbs of the measurement target person on the basis of the heat quantity calculated by the heat quantity calculation unit 221.

The deep body temperature prediction unit 20 includes a first control unit 222 that sets a state of the measurement target person as a resting state instead of using the information in the calculation parameter acquisition unit 1 during the first preliminary calculation, sets the fixed value of the temperature and the fixed value of the humidity corresponding to the temperature neutral range instead of using the information in the calculation parameter acquisition unit 1, causes the heat quantity calculation unit 221 to calculate the heat quantity, and causes the temperature calculation unit 220 to calculate the skin temperature and the deep part temperature, a second control unit 223 that sets a state of the measurement target person as a resting state instead of using the information in the calculation parameter acquisition unit 1 during the second preliminary calculation, sets calculation results from the temperature calculation unit 220 during the first preliminary calculation as an initial value of the skin temperature and an initial value of the deep part temperature of each of the trunk and the limbs, causes the heat quantity calculation unit 221 to calculate the heat quantity, and causes the temperature calculation unit 220 to calculate the skin temperature and the deep part temperature, and a third control unit 224 that sets calculation results from the temperature calculation unit 220 during the second preliminary calculation as an initial value of the skin temperature and an initial value of the deep part temperature of each of the trunk and the limbs during the main calculation, causes the heat quantity calculation unit 221 to calculate the heat quantity on the basis of the information in the calculation parameter acquisition unit 1, and causes the temperature calculation unit 220 to calculate the skin temperature and the deep part temperature.

The calorific value calculation unit 213, the metabolic rate calculation unit 214, the heat transfer/heat radiation amount calculation unit 215, the skin transpiration amount calculation unit 216, the exhalation transpiration amount setting unit 217, the heat exchange amount calculation unit 218, and the heat exchange amount calculation unit 219 configure the heat quantity calculation unit 221 that calculates a heat quantity flowing into and out of the deep part and the skin of a first site and a second site of the measurement target person.

The first measurement unit 211 measures a temperature in the vicinity of the measurement target person (the temperature of the atmosphere of the measurement target person). The second measurement unit 212 measures a humidity in the vicinity of the measurement target person (the humidity of the atmosphere of the measurement target person).

The heat quantity calculation unit 221 calculates a heat quantity flowing into and out of deep parts of the trunk (first site) and the limbs (second site) and the skin of the measurement target person on the basis of the information stored in advance in the calculation parameter acquisition unit 1, the temperature in the vicinity of the measurement target person, and the humidity in the vicinity of the measurement target person.

The temperature calculation unit 220 calculates a skin temperature and a deep part temperature of each of the trunk and the limbs of the measurement target person on the basis of the heat quantity calculated by the heat quantity calculation unit 221.

The first control unit 222 sets a state of the measurement target person as a resting state during the first preliminary calculation, sets a fixed value of the temperature and a fixed value of the humidity corresponding to the temperature neutral range instead of the information stored in the calculation parameter acquisition unit 1 or the measurement results from the measurement units 211 and 212, causes the heat quantity calculation unit 221 to calculate the heat quantity, and causes the temperature calculation unit 220 to calculate the skin temperature and the deep part temperature.

The second control unit 223 sets a state of the measurement target person as a resting state during the second preliminary calculation, sets calculation results from the temperature calculation unit 220 during the first preliminary calculation as an initial value of the skin temperature and an initial value of the deep part temperature of each of the trunk and the limbs, causes the heat quantity calculation unit 221 to calculate the heat quantity, and causes the temperature calculation unit 220 to calculate the skin temperature and the deep part temperature.

The third control unit 224 sets calculation results from the temperature calculation unit 220 during the second preliminary calculation as an initial value of the skin temperature and an initial value of the deep part temperature of each of the trunk and the limbs during the main calculation, causes the heat quantity calculation unit 221 to calculate the heat quantity on the basis of the information stored in the calculation parameter acquisition unit 1 or the measurement results from the measurement units 211 and 212, and causes the temperature calculation unit 220 to calculate the skin temperature and the deep part temperature.

Deep body temperature prediction method: method for calculating deep body temperature change for each time step.

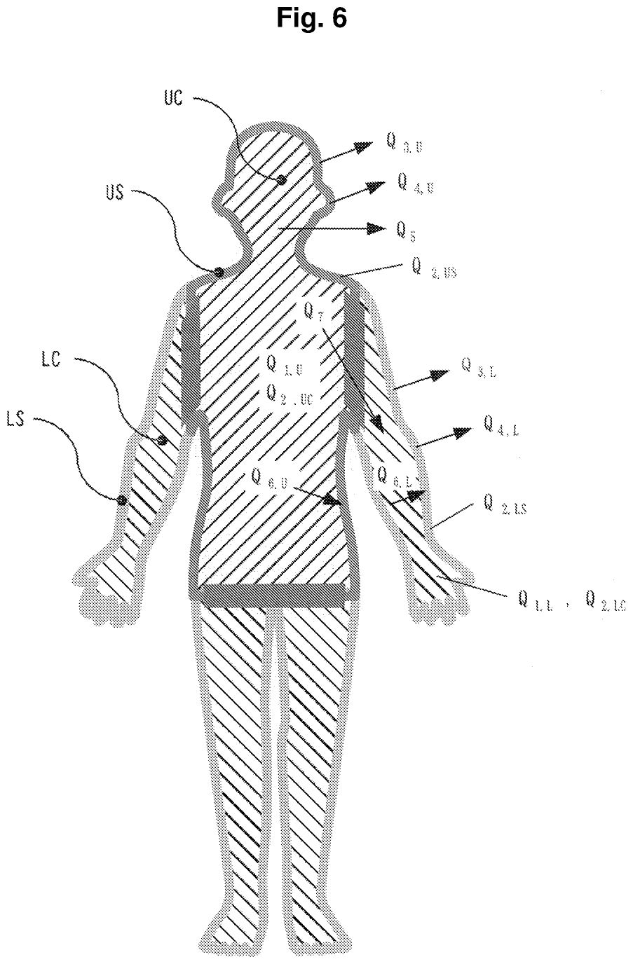

In the present embodiment, as shown in FIG. 6, it is assumed that the body of the measurement target person is composed of two sites such as a trunk U and limbs L, and the two sites such as the trunk U and the limbs L each have two layers such as a deep layer C and a skin layer S. That is, the body of the measurement target person includes a trunk deep layer UC, a trunk skin layer US, a limb deep layer LC, and a limb skin layer LS. In the present embodiment, a heat quantity that flows into and out of each site/each layer of the measurement target person and changes from moment to moment is calculated on the basis of a temperature and a humidity to be acquired and information stored in advance in the calculation parameter acquisition unit 1, and temperature change of each site/each layer of the measurement target person is predicted from a change in the heat quantity.

In the present embodiment, a method of calculating a deep body temperature change for each time step will be described. In the present embodiment, as described above, a calculation example in a case where the body of the measurement target person is regarded as including two sites such as the trunk and the limbs is described, but the trunk may be replaced with the upper body and the limbs may be replaced with the lower body. That is, the body of the measurement target person may be composed of any two sites such as a first site and a second site.

Exemplary equations for predicting a temperature change of the trunk skin layer US, a temperature change of the trunk deep layer UC, a temperature change of the limb skin layer LS, and a temperature change of the limb deep layer LC of the measurement target person are shown in Equations 5 to 8, respectively. T_{US}[t] is the temperature [° C.] of the trunk skin layer US at time t, T_{UC}[t] is the temperature [° C.] of the trunk deep layer UC at time t, T_{LS}[t] is the temperature [° C.] of the limb skin layer LS at time t, and T_{LC}[t] is the temperature [° C.] of the limb deep layer LC at time t.

$$Equation 5$$

$${T}_{US} [ t + Δ t ] = {T}_{US} [ t ] + \frac{{Q}_{2 , US} - {Q}_{3 , U} - {Q}_{4 , U} + {Q}_{6 , U}}{{WC}_{US}} Δ t \qquad ( 5 )$$

$$Equation 6$$

$${T}_{UC} [ t + Δ t ] = {T}_{UC} [ t ] + \frac{{Q}_{1 , U} + {Q}_{2 , UC} - {Q}_{5} - {Q}_{6 , U} - {Q}_{7}}{{WC}_{UC}} Δ t \qquad ( 6 )$$

$$Equation 7$$

$${T}_{LS} [ t + Δ t ] = {T}_{LS} [ t ] + \frac{{Q}_{2 , LS} - {Q}_{3 , L} - {Q}_{4 , L} + {Q}_{6 , L}}{{WC}_{LS}} Δ t \qquad ( 7 )$$

$$Equation 8$$

$${T}_{LC} [ t + Δ t ] = {T}_{LC} [ t ] + \frac{{Q}_{1 , L} + {Q}_{2 , LC} - {Q}_{6 , L} + {Q}_{7}}{{WC}_{LC}} Δ t \qquad ( 8 )$$

Q_{1,U} is a calorific value [W] of the trunk U due to work of the measurement target person, and Q_{1,L} is a calorific value [W] of the limbs L due to the work. Q_{2,US} is a metabolic rate [W] of the trunk skin layer US of the measurement target person, Q_{2,UC} is a metabolic rate [W] of the trunk deep layer UC, Q_{2,LS} is a metabolic rate [W] of the limb skin layer LS, and Q_{2,LC} is a metabolic rate [W] of the limb deep layer LC. Q_{3,U} (first heat exchange amount) is a heat transfer/heat radiation amount [W] between the skin and the outside air in the trunk U of the measurement target person, and Q_{3,L} (first heat exchange amount) is a heat transfer/heat radiation amount [W] between the skin and the outside air in the limbs L.

Q_{4,U} is a skin transpiration amount in the trunk U of the measurement target person, and Q_{4,L} is a skin transpiration amount in the limbs L. Q₅ (fourth heat exchange amount) is an exhalation transpiration amount [W] of the measurement target person. Q_{6,U} (second heat exchange amount) is a heat exchange amount [W] between the deep part and the skin in the trunk U of the measurement target person, and Q_{6,L} (second heat exchange amount) is a heat exchange amount [W] between the deep part and the skin in the limbs L. Q₇ (third heat exchange amount) is a heat exchange amount [W] between the trunk and the limbs in the deep part of the measurement target person.

WC_{US} is a heat capacity [J/° C.] of the trunk skin layer US of the measurement target person, WC_{UC} is a heat capacity [J/° C.] of the trunk deep layer UC, WC_{LS} is a heat capacity [J/° C.] of the limb skin layer LS, and WC_{LC} is a heat capacity [J/° C.] of the limb deep layer LC. Δt is a calculation step time, and is, for example, 1[s] or less.

An average skin temperature Tₛₖ[t+Δt] and a deep body temperature T[t+Δt] at time t+Δt are expressed by Equations 9 and 10, respectively.

$$Equation 9$$

$${T}_{sk} [ t + Δ t ] = sf\_conf \_US \times {T}_{US} [ t + Δ t ] + sf\_conf \_LS \times {T}_{LS} [ t + Δ t ] \qquad ( 9 )$$

$$Equation 10$$

$$T [ t + Δ t ] = {T}_{UC} [ t + Δ t ] \qquad ( 10 )$$

sf_conf_US is a proportion [%] of a surface area of the trunk U to the entire body surface of the measurement target person, and sf_conf_LS is a proportion [%] of a surface area of the limbs L to the entire body surface. The heat capacities WC_{US}, WC_{UC}, WC_{LS}, and WC_{L}C, and the proportions sf_conf_US and sf_conf_LS are known values, and may be actual values, and are stored in advance in the calculation parameter acquisition unit 1 as the information regarding the physical features of the measurement target person.

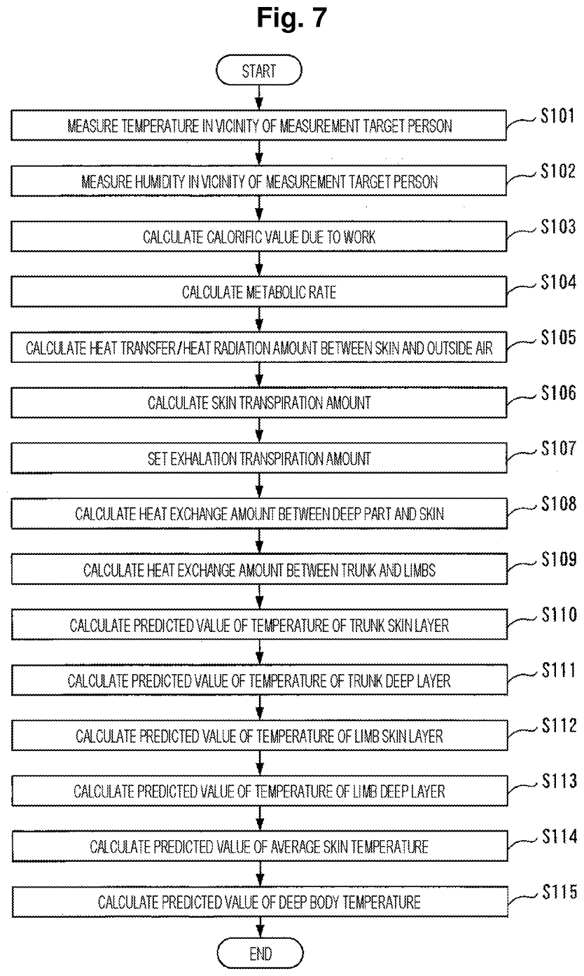

An operation of the deep body temperature prediction unit 20 of the present embodiment includes main calculation and preliminary calculation performed prior to the main calculation. First, the main calculation as a basis will be described. FIG. 7 is a flowchart for describing an operation of the deep body temperature prediction unit 20 of the present embodiment during the main calculation.

The first measurement unit 211 measures a temperature in the vicinity of the measurement target person (the temperature of the atmosphere of the measurement target person) (step S101 in FIG. 7). The second measurement unit 212 measures the humidity in the vicinity of the measurement target person (the humidity of the atmosphere of the measurement target person) (step S102 in FIG. 7).

Calculation of calorific value Q₁ due to work.

Next, the calorific value calculation unit 213 calculates the calorific values Q_{1,U} [W] in the deep layer of the trunk U due to work of the measurement target person and the calorific values Q_{1,L} [W] in the deep layer of the limbs L due to the work according to Equations 11 and 12, respectively, on the basis of the information stored in advance in the calculation parameter acquisition unit 1 (step S103 in FIG. 7).

$$Equation 11$$

$${Q}_{1 , U} = ( METs [ t ] - 1 ) \times 1.05 \times weight \times 4184 \div 3600 \times ex\_conf \_U \qquad ( 11 )$$

$$Equation 12$$

$${Q}_{1 , L} = ( METs [ t ] - 1 ) \times 1.05 \times weight \times 4184 \div 3600 \times ex\_conf \_L \qquad ( 12 )$$

Equations 11 and 12 are disclosed in ““Exercise Guidance for Health 2006˜For the Prevention of Lifestyle Diseases˜”, the Ministry of Health, Labour and Welfare, 2006, <https://www.mhlw.go.jp/shingi/2006/07/dl/so719-3c.pdf>”.

weight is a weight [kg] of the measurement target person, ex_conf_U is a proportion [%] of the muscle mass of the trunk U to the whole body of the measurement target person, and ex_conf_L is a proportion [%] of the muscle mass of the limbs L to the whole body. Each of the weight, the proportions ex_conf_U and ex_conf_L, and METs[t] are known values, and may be actual values, and are stored in advance in the calculation parameter acquisition unit 1 as the information regarding the physical features of the measurement target person.

Calculation of metabolic rate Q₂.

Next, the metabolic rate calculation unit 214 calculates the metabolic rate Q_{2,US} [W] of the trunk skin layer US, the metabolic rate Q_{2,UC} [W] of the trunk deep layer UC, the metabolic rate Q_{2,LS} [W] of the limb skin layer LS, and the metabolic rate Q_{2,LC} [W] of the limb deep layer LC of the measurement target person as shown in Equations 13 to 16, respectively, on the basis of the predicted value Tₛₖ[t][° C.] of an average skin temperature of the measurement target person and the predicted value T[t][° C.] of the deep body temperature at time t calculated by the temperature calculation unit 220 (step S104 in FIG. 7).

$$Equation 13$$

$${Q}_{2 , US} = ( volume\_US \times {A}_{skin} ) \times 1 . {1}^{{T}_{sk} [ t ] - {T}_{sk 0}} \qquad ( 13 )$$

$$Equation 14$$

$${Q}_{2 , UC} = ( \frac{weight \_U}{weight\_ U + weight\_ L} \times M - volume\_US \times {A}_{skín} ) \times 1 . {1}^{T [ t ] - {T}_{0}} \qquad ( 14 )$$

$$Equation 15$$

$${Q}_{2 , LS} = ( volume\_LS \times {A}_{skin} ) \times 1 . {1}^{{T}_{sk} [ t ] - {T}_{sk 0}} \qquad ( 15 )$$

$$Equation 16$$

$${Q}_{2 , LC} = ( \frac{weight\_ L}{weight\_ U + weight\_ L} \times M - volume\_LS \times {A}_{skin} ) \times 1 . {1}^{T [ t ] - {T}_{0}} \qquad ( 16 )$$

Equations 13 to 16 are disclosed in “Ronald J Spiegel, “A Review of Numerical Models for Predicting the Energy Deposition and Resultant Thermal Response of Humans Exposed to Electromagnetic Fields”, IEEE Transactions on Microwave Theory and Techniques, Volume 32, Issue 8, 1984”.

Aₛₖᵢₙ is a constant related to metabolism, volume_US is a volume [m³] of the trunk skin layer US of the measurement target person, volume_LS is the volume [m³] of the limb skin layer LS, weight_U is a weight [kg] of the trunk U of the measurement target person, and weight_L is a weight [kg] of the limbs L. The constants Aₛₖᵢₙ, the volumes volume_US and volume_LS, and the weights weight_U and weight_L are known values, and may be actual values, and are stored in advance in the calculation parameter acquisition unit 1 as the information regarding the physical features of the measurement target person.

Tₛₖₒ is a reference temperature of an average skin temperature at which perspiration of the measurement target person starts, and T₀ is a reference temperature of a deep body temperature at which perspiration starts. As will be described later, the initial value Tₛₖ[O] of the average skin temperature, the initial value T[O] of the deep body temperature, the initial value T_{US}[O] of the temperature of the trunk skin layer US, the initial value T_{UC}[O] of the temperature of the trunk deep layer UC, the initial value T_{LS}[O] of the temperature of the limb skin layer LS, and the initial value T_{LC}[O] of the temperature of the limb deep layer LC are set by the first control unit 222, the second control unit 223, and the third control unit 224. In the first calculation at time t=O, Tₛₖ[t]=Tₛₖ[O], T[t]=T[O], T_{US}[t]=T_{US}[O], T_{UC}[t]=T_{UC}[O], T_{Ls}[t]=T_{LS}[O], and T_{LC}[t]=T_{LC}[O].

The metabolic rate calculation unit 214 calculates M in Equations 14 and 16 as in Equation 17.

$$Equation 17$$

$$M = ( 0.1238 + 0.0 \; 481 \times weight\_ C + 0.0 \; 234 \times height \text{} - 0.0138 \times age - sexcoef ) \times {10}^{6} \times 24 \div 3600 \times activity\_level \times {A}_{coef} \qquad ( 17 )$$

Equations 17 is disclosed in “AA Ganpule, et al., “Interindividual variability in sleeping metabolic rate in Japanese subjects”, European Journal of Clinical Nutrition, volume 61, 2007”. weight_C is a weight [kg] of the deep part of the measurement target person, height is a height [cm] of the measurement target person, and age is the age of the measurement target person. sexcoef is a constant of 0.5473 in a case where the measurement target person is a male and 0.5473×2 in a case where the measurement target person is a female.

activity_level is a physical activity level, and A_{coef} is a parameter for metabolic adjustment. The weight weight_C, the height height, the age age, the constant sexcoef, and the parameter A_{coef} are known values, and may be actual values, and are stored in advance in the calculation parameter acquisition unit 1 as the information regarding the physical features of the measurement target person. The constant activity_level is stored in advance in the calculation parameter acquisition unit 1 as the information regarding the work conditions of the measurement target person.

As an example of setting activity_level, activity_level may be set to about 1.5 in a case where most of the life of the measurement target person is sedentary and usually performs static activity. In a case where the measurement target person performs sitting-centered work, but includes movement in the workplace, work in a standing position, serving a customer, or the like, or includes any of commuting, shopping, housework, light sports, and the like, activity_level may be set to about 1.75. In a case where the measurement target person is a worker who moves or stands up a lot, or has an active exercise habit in leisure such as sports, activity_level may be set to about 2.0. As described above, activity_level may be appropriately set according to a work situation of the measurement target person.

Calculation of heat transfer/heat radiation amount Q₃ between skin and outside air.

Next, the heat transfer/heat radiation amount calculation unit 215 calculates the heat transfer/heat radiation amount Q_{3,U} [W] between the skin and the outside air in the trunk U and the heat transfer/heat radiation amounts Q_{3,L} [W] between the skin and the outside air in the limbs L of the measurement target person on the basis of the temperature Tₐ[t][° C.] in the vicinity of the measurement target person measured by the first measurement unit 211, and the predicted value Tₛₖ[t][° C.] of the average skin temperature of the measurement target person, the predicted value T_{US}[t][° C.] of the temperature of the trunk skin layer US, and the predicted value T_{LS}[t][° C.] of the temperature of the limb skin layer LS at time t, which are calculated by the temperature calculation unit 220 (step S105 in FIG. 7). In a case where the first measurement unit 211 measures the air temperature Tₐ[t][° C.] outside the clothing of the measurement target person, the heat transfer/heat radiation amounts Q_{3,U} and Q_{3,L} are expressed by Equations 18 and 19.

$$Equation 18$$

$${Q}_{3 , U} = sf\_conf \_US \times fcl\_US \times HS \times ( {T}_{US} [ t ] - {T}_{a} [ t ] ) \qquad ( 18 )$$

$$Equation 19$$

$${Q}_{3 , L} = sf\_conf \_LS \times ( fcl\_LS \times coverage + 1 - coverage ) \times HS \times ( {T}_{LS} [ t ] - {T}_{a} [ t ] ) \qquad ( 19 )$$

The proportions sf_conf_US and sf_conf_LS are as described above. fcl_US is a constant representing the heat transfer efficiency of the trunk U by the clothing, fcl_LS is a constant representing the heat transfer efficiency of the limbs L by the clothing, and coverage is a proportion [%] at which the limbs L are covered with the clothing. The constants fcl_US and fcl_LS, and the proportion coverage are known values, and are stored in advance in the calculation parameter acquisition unit 1 as the information regarding the work conditions of the measurement target person.

HS is a heat exchange coefficient [W/° C.] of the measurement target person with the air. The heat transfer/heat radiation amount calculation unit 215 calculates the heat exchange coefficient HS[W/° C.] according to Equation 20.

$$Equation 20$$

$$HS = ( {H}_{cm} + {H}_{r} ) \times sf \qquad ( 20 )$$

sf is a body surface area [m²] of the measurement target person. The heat transfer/heat radiation amount calculation unit 215 can calculate the body surface area sf [m²] from the weight weight [kg] and the height height [m] of the measurement target person. Examples of the calculation formula of the body surface area sf [m²] include prediction formulas such as DuBois's formula and Fujimoto's formula.

H_{cm} is a convective heat transfer coefficient [W/° C./m²] and Hᵣ is a radiative heat transfer coefficient [W/° C./m²]. The heat transfer/heat radiation amount calculation unit 215 calculates the convective heat transfer coefficient H_{cm} [W/° C./m²] and the radiative heat transfer coefficient Hᵣ[W/° C./m²] according to Equations 21 and 22, respectively.

$$Equation 21$$

$${H}_{cm} = \sqrt{4.63 \times \sqrt{\vert {T}_{sk} [ t ] - {T}_{a} [ t ] \vert} + 199.74 \times {V}_{air} - 9.80} \qquad ( 21 )$$

$$Equation 22$$

$${H}_{r} = ( 5.67 \times 1 {0}^{- 8} ) \times 0.99 \times 0.93 \times 0.794 \times \{ {( {T}_{sk} [ t ] + 273 )}^{2} + {( {T}_{a} [ t ] + 273 )}^{2} \} \times \{ ( {T}_{sk} [ t ] + 273 ) + ( {T}_{a} [ t ] + 273 ) \} \qquad ( 22 )$$

Equations 21 and 22 are disclosed in “D. Fiala, et al., “A computer model of human thermoregulation for a wide range of environmental conditions: the passive system”, Journal of Applied Physiology, 1985”. Vₐᵢᵣ is a wind speed [m/s]. The heat transfer/heat radiation amount calculation unit 215 may use, as the wind speed Vₐᵢᵣ[m/s], an actually measured value measured by an anemometer or the like, or a known value of a wind speed in the clothing disclosed in documents or the like. In the present embodiment, a value stored in advance in the calculation parameter acquisition unit 1 is used as the wind speed Vₐᵢᵣ[m/s].

Calculation of skin transpiration amount Q4.

Next, the skin transpiration amount calculation unit 216 calculates the skin transpiration amounts Q_{4,U} [W] in the trunk U and the skin transpiration amounts Q_{4,L} [W] in the limbs L of the measurement target person according to Equations 23 and 24, respectively, on the basis of the temperature Tₐ[t][° C.] in the vicinity of the measurement target person measured by the first measurement unit 211, the relative humidity humidity[t][%] in the vicinity of the measurement target person measured by the second measurement unit 212, and the predicted value Tₛₖ[t][° C.] of the average skin temperature and the predicted value T[t][° C.] of the deep body temperature at time t calculated by the temperature calculation unit 220 (step S106 in FIG. 7).

$$Equation 23$$

$${Q}_{4 , U} = sw\_conf \_US \times sw \qquad ( 23 )$$

$$Equation 24$$

$${Q}_{4 , L} = sw\_conf \_LS \times sw \qquad ( 24 )$$

sw_conf_US is a proportion [%] of an amount of perspiration of the trunk U to the entire body surface of the measurement target person, and sw_conf_LS is a proportion [%] of an amount of perspiration of the limbs L to the entire body surface. The proportions sw_conf_US and sw_conf_LS are known values, and may be actual values, and are stored in advance in the calculation parameter acquisition unit 1 as the information regarding the physical features of the measurement target person. sw is a skin transpiration amount [W] of the whole body. The skin transpiration amount calculation unit 216 may calculate the skin transpiration amount sw [W] according to Equation 25.

$$\begin{matrix}Equation 25 & \\ sw = min ( E , {E}_{max} ) & ( 25 )\end{matrix}$$

min (E, Eₘₐₓ) means that a smaller one of E and Eₘₐₓ is employed. E is a sum [W] of insensitive transpiration and sensitive transpiration in the skin of the measurement target person. The skin transpiration amount calculation unit 216 may calculate the sum E [W] of insensitive transpiration and sensitive transpiration according to Equation 26.

$$\begin{matrix}Equation 26 & \\ E = P I + {Q}_{e v} \times swrate \div 60 & ( 26 )\end{matrix}$$

PI is insensitive transpiration [W] in the skin of the measurement target person, and Qₑᵥ is evaporation heat [J/g] of water. The insensitive transpiration PI [W] is a known value, and may be an actual value, and is stored in advance in the calculation parameter acquisition unit 1 as the information regarding the physical features of the measurement target person. The evaporation heat Qₑᵥ [J/g] of water is a known value, and is stored in advance in the calculation parameter acquisition unit 1 as the information regarding the environmental conditions of the measurement target person. swrate is sensitive transpiration [g/min]. The skin transpiration amount calculation unit 216 may calculate the sensitive transpiration swrate [g/min] according to Equation 27.

$$\begin{matrix}Equation 27 & \\ swrate = ( a 11 \times tanh ( b 11 \times ( {T}_{s k} [ t ] - {T}_{s k 0} ) - b 10 ) + a 10 ) \times ( {T}_{s k} [ t ] - {T}_{s k 0} ) + ( a 21 \times tanh ( b 21 \times ( T [ t ] - {T}_{0} ) - b 20 ) + a 20 ) \times ( T [ t ] - {T}_{0} ) & ( 27 )\end{matrix}$$

Equation 27 is disclosed in “D. Fiala, et al., “Computer prediction of human thermoregulatory and temperature responses to a wide range of environmental conditions”, International Journal of Biometeorology, volume 45, 2001”. aij and bij (where i=1 and 2, and j=0 and 1) are perspiration coefficients. Each of the perspiration coefficients aij and bij is a known value, and may be an actual value according to the ease of sweating of the measurement target person, and is stored in advance in the calculation parameter acquisition unit 1 as the information regarding the physical features of the measurement target person. Specifically, the perspiration coefficients aij and bij may be set as shown in Equation 28 according to three levels such as low (hard to sweat), normal (normal), and high (easy to sweat).

$$\begin{matrix}Equation 28 & \\ [ \begin{matrix}a 10 & a 11 & a 20 & a 21 & b 10 & b 11 & b 20 & b 21\end{matrix} ] = \{ \begin{matrix}[ \begin{matrix}0.95 & 0.55 & 3.8 & 3.2 & 0.09 & 0.59 & 1.8 & 2.7\end{matrix} ] & ( low ) \\ [ \begin{matrix}1.2 & 0.8 & 6.3 & 5.7 & 0.19 & 0.59 & 1.03 & 1.98\end{matrix} ] & ( normal ) \\ [ \begin{matrix}1.35 & 0.95 & 7.3 & 6.7 & 0.15 & 0.59 & 0.47 & 2.3\end{matrix} ] & ( high )\end{matrix} & ( 28 )\end{matrix}$$

On the other hand, Eₘₐₓ is the maximum evaporation heat [W]. In a case where the first measurement unit 211 measures the air temperature Tₐ[t][° C.] outside the clothing of the measurement target person and the second measurement unit 212 measures the relative humidity humidity[t][%] outside the clothing of the measurement target person, the skin transpiration amount calculation unit 216 may calculate the maximum evaporation heat Eₘₐₓ[W] according to Equation 29.

$$\begin{matrix}Equation 29 & \\ {E}_{max} = {E}_{max \_ coef} \times {H}_{c} \times ( {P}_{s} - humidity [ t ] \times {P}_{a} ) \times ( sw\_conf \_US \times fpcl\_US + sw\_conf \_LS \times ( fpcl\_LS \times coverage + 1 - coverage ) ) & ( 29 )\end{matrix}$$

The proportions sw_conf_US and sw_conf_LS, and the coverage are as described above. fpcl_US is a constant representing a heat transfer efficiency by clothing in the trunk U of the measurement target person, fpcl_LS is a constant representing a heat transfer efficiency by clothing in the limbs L, and E_{max_coef} is a constant related to the maximum evaporation heat. The constants fpcl_US, fpcl_LS, and E_{max_coef} are known values, and may be actual values, and are stored in advance in the calculation parameter acquisition unit 1 as the information regarding the work conditions of the measurement target person.

Hₑ is heat transfer [W·m²/° C.] by convection depending on the wind speed of the air. The skin transpiration amount calculation unit 216 may calculate the heat transfer Hₑ[W·m²/° C.] according to Equation 30.

$$\begin{matrix}Equation 30 & \\ {H}_{c} = 3.0 \times \sqrt{10 \times {V}_{a i r}} & ( 30 )\end{matrix}$$

Equations 29 and 30 are disclosed in “I. Laakso, et al., “Dominant factors affecting temperature rise in simulations of human thermoregulation during RF exposure”, Physics in Medicine and Biology, Volume 56, 2011”. Pₛ is the saturated water vapor pressure [kPa] in the skin layer of the measurement target person. The skin transpiration amount calculation unit 216 may calculate the saturated water vapor pressure Pₛ[kPa] according to Equation 31.

$$\begin{matrix}Equation 31 & \\ {P}_{s} = 7.5 \times {10}^{7.23 - \frac{1750.3}{273 + {T}_{sk} [ t ] - 38.1}} & ( 31 )\end{matrix}$$

Pₐ is saturated water vapor pressure [kPa] in the atmosphere in which the humidity is measured. The skin transpiration amount calculation unit 216 may calculate the saturated water vapor pressure Pₐ[kPa] according to Equation 32.

$$\begin{matrix}Equation 32 & \\ {P}_{a} = 7.5 \times {10}^{7.23 - \frac{1750.3}{273 + {T}_{a} [ t ] - 38.1}} & ( 32 )\end{matrix}$$

Determination of exhalation transpiration amount Q₅

Next, the exhalation transpiration amount setting unit 217 sets the transpiration amount Q₅ [W] by the exhalation of the measurement target person as in Equation 33 (step S107 in FIG. 7). The exhalation transpiration amount Q₅ [W] corresponds to a heat exchange amount between the outside air and the deep part of the measurement target person.

$$\begin{matrix}Equation 33 & \\ {Q}_{5} = 9.54 & ( 33 )\end{matrix}$$

Calculation of heat exchange amount Q₆ between deep part and skin.

Next, the heat exchange amount calculation unit 218 calculates the heat exchange amount Q_{6,U} [W] between the deep part and the skin in the trunk U of the measurement target person and the heat exchange amount Q_{6,L} [W] between the deep part and the skin in the limbs L according to Equations 34 and 35, respectively, on the basis of the information stored in advance in the calculation parameter acquisition unit 1, the predicted value T_{US}[t][° C.] of the temperature of the trunk skin layer US, the predicted value T_{UC}[t][° C.] of the temperature of the trunk deep layer UC, the predicted value T_{LS}[t][° C.] of the temperature of the limb skin layer LS, the predicted value T_{LC}[t][° C.] of the temperature of the limb deep layer LC, the predicted value Tₛₖ[t][° C.] of the average skin temperature, and the predicted value T[t][° C.] of the deep body temperature at time t, calculated by the temperature calculation unit 220 (step S108 in FIG. 7).

$$\begin{matrix}Equation 34 & \\ {Q}_{6 , U} = sf\_conf \_US \times hx \times ( {T}_{U C} [ t ] - {T}_{U S} [ t ] ) & ( 34 )\end{matrix}$$

$$\begin{matrix}Equation 35 & \\ {Q}_{6 , L} = sf\_conf \_LS \times hx \times ( {T}_{L C} [ t ] - {T}_{L S} [ t ] ) & ( 35 )\end{matrix}$$

The proportions sf_conf_US and sf_conf_LS are as described above. hx is a heat exchange coefficient between the skin and the deep part of the measurement target person. The heat exchange amount calculation unit 218 may calculate the heat exchange coefficient hx according to Equation 36.

$$\begin{matrix}Equatiion 36 & \\ hx = min \{ e \times hx 0 + ( 1 - e ) \times hx 0 \times hx {1}^{T [ n ] - {T}_{0} + \frac{{T}_{s k} [ t ] - {T}_{sk 0}}{a} + \frac{METs [ t ] - 1}{b}} , hx\_max \} & ( 36 )\end{matrix}$$

METs[t] is as described above. a, b, e, hx0, hx1, and hx_max are parameters related to the heat exchange coefficient. The parameters a, b, e, hx0, hx1, and hx_max are known values, and may be actual values, and are stored in advance in the calculation parameter acquisition unit 1 as the information regarding the physical features of the measurement target person.

Calculation of heat exchange amount Q₇ between trunk and limbs.

Next, the heat exchange amount calculation unit 219 calculates the heat exchange amount Q₇ [W] between the trunk U and the limbs L in the deep part of the measurement target person according to Equation 37 on the basis of the predicted value T_{UC}[t][° C.] of the temperature of the trunk deep layer UC, the temperature T_{LC}[t][° C.] of the limb deep layer LC, the predicted value Tₛₖ[t][° C.] of the average skin temperature, and the predicted value T[t][° C.] of the deep body temperature at time t, calculated by the temperature calculation unit 220 (step S109 in FIG. 7).

$$\begin{matrix}Equation 37 & \\ {Q}_{7} = h c c \times ( {T}_{L C} [ t ] - {T}_{U C} [ t ] ) & ( 37 )\end{matrix}$$

hcc is a heat exchange coefficient between the trunk U and the limbs L in the deep part of the measurement target person. The heat exchange amount calculation unit 219 may calculate the heat exchange coefficient hcc according to Equation 38.

$$\begin{matrix}Equation 38 & \\ hcc = f \times hcc 0 + ( 1 - f ) \times hcc 0 \times hcc\_T \times hcc\_M & ( 38 )\end{matrix}$$

f and hcc0 are parameters related to the heat exchange coefficient. The parameters f and hcc0 are known values, and may be actual values, and are stored in advance in the calculation parameter acquisition unit 1 as the information regarding the physical features of the measurement target person. hcc_T is the temperature contribution of the heat exchange coefficient hcc, and hcc_M is the METs contribution of the heat exchange coefficient hcc. The heat exchange amount calculation unit 219 may calculate the temperature contribution hcc_T of the heat exchange coefficient hcc according to Equation 39.

$$\begin{matrix}Equation 39 & \\ hcc\_T = min \{ hcc {1}^{T [ t ] - {T}_{0} + \frac{{T}_{s k} [ t ] - {T}_{s k 0}}{a}} , hcc\_Tmax \} & ( 39 )\end{matrix}$$

hcc_Tmax is a prescribed upper limit value of hcc_T. The heat exchange amount calculation unit 219 may calculate the METs contribution hcc_M of the heat exchange coefficient hcc according to Equation 40.

$$\begin{matrix}Equation 40 & \\ hcc\_M = min \{ hcc {1}^{\frac{aveMETs [ t ] - 1}{b}} , hcc\_Mmax \} & ( 40 )\end{matrix}$$

hcc_Mmax is a prescribed upper limit value of hcc_M. hcc1 is a parameter related to a heat exchange coefficient. The upper limit values hcc_Tmax and hcc_Mmax, and the parameter hcc1 are known values, and may be actual values, and are stored in advance in the calculation parameter acquisition unit 1 as the information regarding the physical features of the measurement target person. aveMETs[t] is a time average value of the number of METs, and in the present embodiment, the same value as METs[t] may be used. The parameters a and b are as described above.

As described above, it is possible to calculate each of the heat quantities Q_{1,U}, Q_{1,L}, Q_{2,US}, Q_{2,UC}, Q_{2,LS}, Q_{2,LC}, Q_{3,U}, Q_{3,L}, Q_{4,U}, Q_{4,L}, Q₅, Q_{6,U}, Q_{6,L}, and Q₇ with the calorific value calculation unit 213, the metabolic rate calculation unit 214, the heat transfer/heat radiation amount calculation unit 215, the skin transpiration amount calculation unit 216, the exhalation transpiration amount setting unit 217, the heat exchange amount calculation unit 218, and the heat exchange amount calculation unit 219.

The temperature calculation unit 220 calculates a predicted value T_{US}[t+Δt][° C.] of the temperature of the trunk skin layer US after Δt elapses according to Equation 5 on the basis of the predicted value T_{US}[t][° C.] of the temperature of the trunk skin layer US of the measurement target person at time t, the metabolic rate Q_{2,US} [W] of the trunk skin layer US, the heat transfer/heat radiation amount Q_{3,U} [W] between the skin and the outside air in the trunk U, the skin transpiration amount Q_{4,U} [W] in the trunk U, and the heat exchange amount Q_{6,U} [W] between the deep part and the skin in the trunk U (step S110 in FIG. 7).

The temperature calculation unit 220 calculates a predicted value T_{UC}[t+Δt][° C.] of the temperature of the trunk deep layer UC after Δt elapses according to Equation 6 on the basis of the predicted value T_{UC}[t][° C.] of the temperature of the trunk deep layer UC of the measurement target person at time t, the calorific value Q_{1,U}[W] in the deep layer of the trunk U, the metabolic rate Q_{2,UC} [W] of the trunk deep layer UC, the exhalation transpiration amount Q₅ [W], the heat exchange amount Q_{6,U} [W] between the deep part and the skin of the trunk U, and the heat exchange amount Q₇ [W] between the trunk U and the limbs L in the deep part (step S111 in FIG. 7).

The temperature calculation unit 220 calculates a predicted value T_{LS}[t+Δt][° C.] of the temperature of the limb skin layer LS after Δt elapses according to Equation 7 on the basis of the predicted value T_{LS}[t][° C.] of the temperature of the limb skin layer LS of the measurement target person at time t, the metabolic rate Q_{2,LS} [W] of the limb skin layer LS, the heat transfer/heat radiation amount Q_{3,L} [W] between the skin and the outside air in the limbs L, the skin transpiration amount Q_{4,L} [W] in the limbs L, and the heat exchange amount Q_{6,L} [W] between the deep part and the skin in the limbs L (step S112 in FIG. 7).

The temperature calculation unit 220 calculates a predicted value T_{LC}[t+Δt][° C.] of the temperature of the limb deep layer LC after Δt elapses according to Equation 8 on the basis of the predicted value T_{LC}[t][° C.] of the temperature of the limb deep layer LC of the measurement target person at time t, the calorific value Q_{1,L} [W] in the deep layer of the limbs L, the metabolic rate Q_{2,LC} [W] of the limb deep layer LC, the heat exchange amount Q_{6,L} [W] between the deep part and the skin in the limbs L, and the heat exchange amount Q₇ [W] between the trunk U and the limbs L in the deep part (step S113 in FIG. 7).

As described above, the predicted values T_{US}[t+Δt], T_{UC}[t+Δt], T_{LS}[t+Δt], and T_{LC}[t+Δt] of the temperature can be sequentially calculated.

Then, the temperature calculation unit 220 calculates a predicted value Tₛₖ[t+Δt][° C.] of the average skin temperature after Δt elapses according to Equation 9 (step S114 in FIG. 7). The temperature calculation unit 220 calculates a predicted value T[t+Δt][° C.] of the deep body temperature after Δt elapses according to Equation 10 (step S115 in FIG. 7).

The deep body temperature prediction unit 20 performs the above processes in steps S101 to S115 for each time Δt. In the calculation for the time after Δt, the processes in steps S1o1 to S115 may be performed with T_{US}[t+Δt], T_{UC}[t+Δt], T_{LS}[t+Δt], T_{LC}[t+Δt], Tₛₖ[t+Δt], and T[t+Δt] calculated in the previous time as T_{US}[t], T_{UC}[t], T_{LS}[t], T_{LC}[t], Tₛₖ[t], and T[t], respectively.

Next, the preliminary calculation performed before the main calculation in steps S101 to S115 will be described. In the present embodiment, after initial temperatures of each site/each layer of the measurement target person in the measurement environment are predicted through the following preliminary calculation, a deep body temperature of the measurement target person is predicted for the future time at every Δt through the main calculation described in steps S101 to S115.

Preliminary calculation I for initial temperature prediction of each site/each layer.

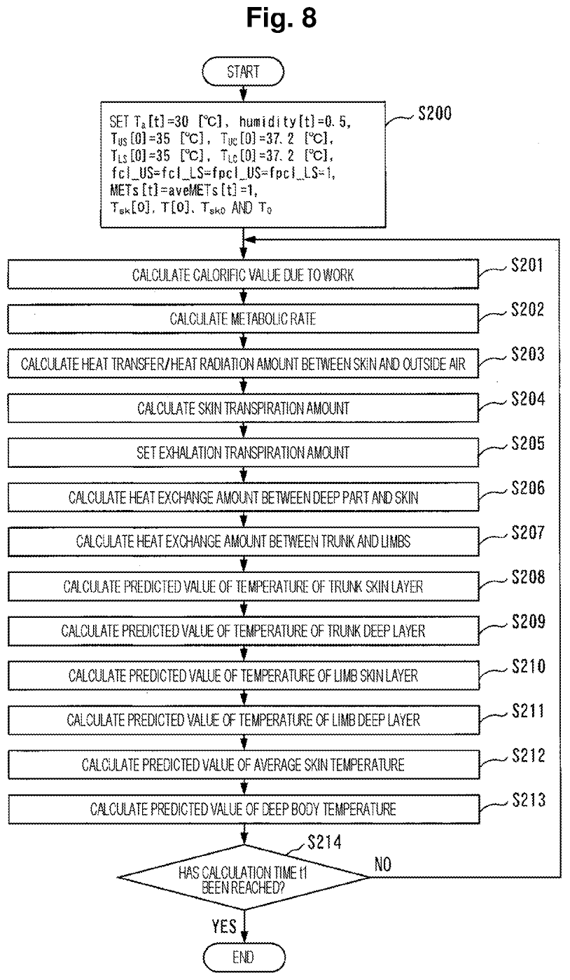

FIG. 8 is a flowchart for describing an operation of the deep body temperature prediction unit 20 of the present embodiment during the preliminary calculation I. In the preliminary calculation I (first preliminary calculation), a process of calculating the above heat quantity and calculating temperatures of each site/each layer is performed for a certain period of time under the condition that a deep body temperature of the measurement target person is constant, the measurement target person is in a resting state, and the measurement target person does not wear clothes under a thermal environment of isotherm and isohumidity of a temperature and a humidity corresponding to a temperature neutral range.

Specifically, the first control unit 222 sets the temperature Tₐ[t] in the vicinity of the measurement target person to 30[° C.], the relative humidity [t] in the vicinity of the measurement target person to 0.5, the initial value T_{US}[O] of the temperature of the trunk skin layer US of the measurement target person to 35[° C.], the initial value T_{UC}[O] of the temperature of the trunk deep layer UC to 37.2[° C.], the initial value T_{LS}[O] of the temperature of the limb skin layer LS to 35[° C.], and the initial value T_{LC}[O] of the temperature of the limb deep layer LC to 37.2[° C.](step S200 in FIG. 8).

The first control unit 222 sets all the constant fcl_US representing the heat transfer efficiency by the clothing of the trunk U, the constant fcl_LS representing the heat transfer efficiency by the clothing of the limbs L, the constant fpcl_US representing the heat transfer efficiency by the clothing in the trunk U, and the constant fpcl_LS representing the heat transfer efficiency by the clothing in the limbs L to 1 (fcl_US=fcl_LS=fpcl_US=fpcl_LS=1), and sets METs[t] and aveMETs[t] to 1 (step S200 in FIG. 8).

The first control unit 222 sets the values calculated with t+Δt=o in Equations 9 and 10 as the initial value Tₛₖ[O][° C.] of the average skin temperature and the initial value T[O][° C.] of the deep body temperature, and sets the reference temperatures Tₛₖₒ=Tₛₖ[O][° C.] and Tₒ=T[O][° C.](step S200 in FIG. 8). The first control unit 222 sets the values described in steps S103 to S115 for the parameters other than the values set in step S200.

Operations of the calorific value calculation unit 213, the metabolic rate calculation unit 214, the heat transfer/heat radiation amount calculation unit 215, the skin transpiration amount calculation unit 216, the exhalation transpiration amount setting unit 217, the heat exchange amount calculation unit 218, the heat exchange amount calculation unit 219, and the temperature calculation unit 220 (steps S201 to S213 in FIG. 8) are similar to those in steps S103 to S115. Settings different from steps S103 to S115 are as described in step S200.

The first control unit 222 executes the processes in steps S201 to S213 for each time Δt until a certain calculation time t=t1 is reached from t=o (YES in step S214 in FIG. 8). The calculation time t1 may be set to a value at which the predicted value Tₛₖ[t+Δt][° C.] of the average skin temperature reaches a steady state.

Preliminary calculation II for initial temperature prediction of each site/each layer.

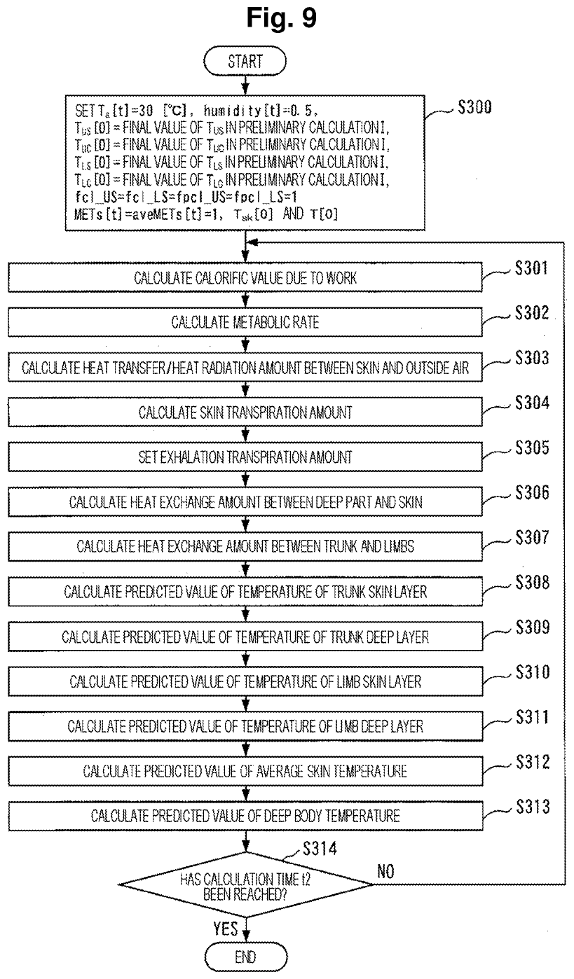

FIG. 9 is a flowchart illustrating an operation of the deep body temperature prediction unit 20 of the present embodiment during the preliminary calculation II. In the preliminary calculation II (first preliminary calculation), the final values of the temperatures T_{US}[t+Δt], T_{UC}[t+Δt], T_{LS}[t+Δt], T_{LC}[t+Δt], Tₛₖ[t+Δt], and T[t+Δt] calculated through the preliminary calculation I are set as initial values of the temperatures of each site/each layer, and a process of calculating the above heat quantity and calculating temperatures of each site/each layer is performed for a certain period of time under the condition that the measurement target person is in a resting state, and the measurement target person does not wear clothes under a thermal environment of isotherm and isohumidity of a temperature and a humidity corresponding to a temperature neutral range.

Specifically, the first control unit 222 sets the temperature Tₐ[t] in the vicinity of the measurement target person to 30[° C.] and the relative humidity in the vicinity of the measurement target person to humidity[t] to 0.5, and sets the final value of T_{US}[t+Δt] in the preliminary calculation I to the initial value T_{US}[O] of the temperature of the trunk skin layer US of the measurement target person, the final value of T_{UC}[t+Δt] in the preliminary calculation I to the initial value T_{UC}[O] of the temperature of the trunk deep layer UC, the final value of T_{LS}[t+Δt] in the preliminary calculation I to the initial value T_{LS}[O] of the temperature of the limb skin layer LS, and the final value of T_{LC}[t+Δt] in the preliminary calculation I to the initial value T_{LC}[O] of the temperature of the limb deep layer LC (step S300 in FIG. 9).

The first control unit 222 sets all the constant fcl_US representing the heat transfer efficiency by the clothing of the trunk U, the constant fcl_LS representing the heat transfer efficiency by the clothing of the limbs L, the constant fpcl_US representing the heat transfer efficiency by the clothing in the trunk U, and the constant fpcl_LS representing the heat transfer efficiency by the clothing in the limbs L to 1 (fcl_US=fcl_LS=fpcl_US=fpcl_LS=1), and sets METs[t] and aveMETs[t] to 1 (step S300 in FIG. 9).

The first control unit 222 sets the values calculated by setting t+Δt=o in Equations 9 and 10 as the initial value Tₛₖ[O] of the average skin temperature and the initial value T[O] of the deep body temperature (step S300 in FIG. 9). The first control unit 222 sets values described in steps S103 to S115 for parameters other than the values set in step S300. The reference temperatures Tₛₖₒ and T₀ are the same as the values set in step S200.

Operations of the calorific value calculation unit 213, the metabolic rate calculation unit 214, the heat transfer/heat radiation amount calculation unit 215, the skin transpiration amount calculation unit 216, the exhalation transpiration amount setting unit 217, the heat exchange amount calculation unit 218, the heat exchange amount calculation unit 219, and the temperature calculation unit 220 (steps S301 to S313 in FIG. 9) are similar to those in steps S103 to S115. Settings different from steps S103 to S115 are as described in step S300.

The first control unit 222 executes the processes in steps S301 to S313 for each time Δt until a certain calculation time t=t2 is reached from t=o (YES in step S314 in FIG. 9). The calculation time t2 may be set to a value at which the predicted value T[t+Δt][° C.] of the deep body temperature reaches a steady state.

Preliminary calculation III for initial temperature prediction of each site/each layer.

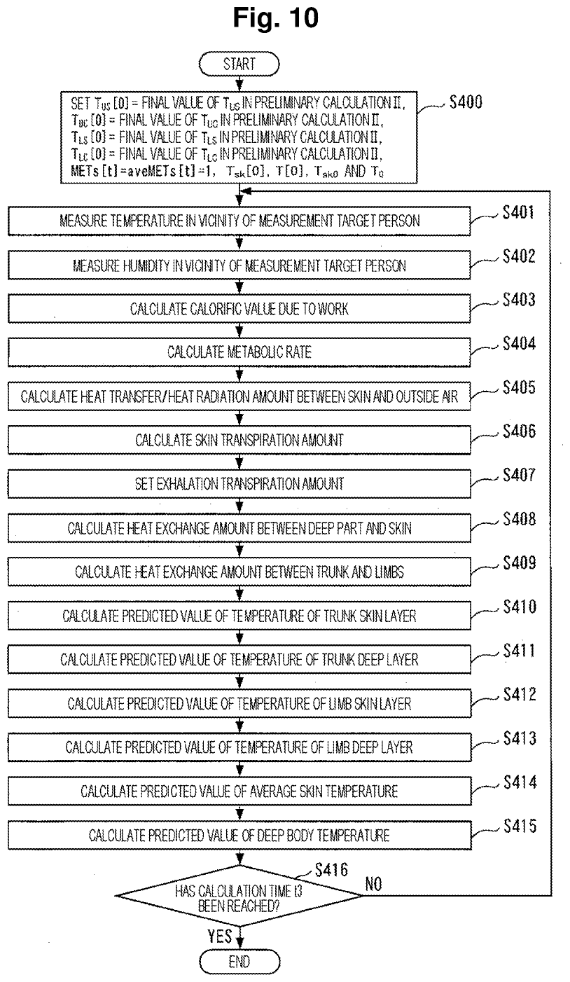

FIG. 10 is a flowchart illustrating an operation of the deep body temperature prediction unit 20 of the present embodiment during the preliminary calculation III. In the preliminary calculation III (second preliminary calculation), the final values of the temperatures T_{US}[t+Δt], T_{UC}[t+Δt], T_{LS}[t+Δt], T_{LC}[t+Δt], Tₛₖ[t+Δt], and T[t+Δt] calculated through the preliminary calculation II are set as initial values of the temperatures of each site/each layer, and a process of calculating the above heat quantity and calculating temperatures of each site/each layer is performed for a certain period of time under the condition that the measurement target person is in a resting state and the measurement target person is wearing clothes.

Specifically, the second control unit 223 sets the final value of T_{US}[t+Δt] in the preliminary calculation II as the initial value T_{US}[O] of the temperature of the trunk skin layer US of the measurement target person, the final value of T_{UC}[t+Δt] in the preliminary calculation II as the initial value T_{UC}[O] of the temperature of the trunk deep layer UC, the final value of T_{LS}[t+Δt] in the preliminary calculation II as the initial value T_{LS}[O] of the temperature of the limb skin layer LS, and the final value of T_{LC}[t+Δt] in the preliminary calculation II as the initial value T_{LC}[O] of the temperature of the limb deep layer LC (step S400 in FIG. 1).

The second control unit 223 sets METs[t] and aveMETs[t] to 1. The second control unit 223 sets the values calculated by setting t+Δt=o in Equations 9 and 10 as the initial value Tₛₖ[O] of the average skin temperature and the initial value T[O] of the deep body temperature, and sets the reference temperature Tₛₖₒ=Tₛₖ[O][° C.] and T₀=T[O][° C.](step S400 in FIG. 1). The second control unit 223 sets the values described in steps S103 to S115 for parameters other than the values set in step S400.

The first measurement unit 211 measures a temperature in the vicinity of the measurement target person (the temperature of the atmosphere of the measurement target person) (step S401 in FIG. 1). The second measurement unit 212 measures a humidity in the vicinity of the measurement target person (the humidity of the atmosphere of the measurement target person) (step S402 in FIG. 1).

Operations of the calorific value calculation unit 213, the metabolic rate calculation unit 214, the heat transfer/heat radiation amount calculation unit 215, the skin transpiration amount calculation unit 216, the exhalation transpiration amount setting unit 217, the heat exchange amount calculation unit 218, the heat exchange amount calculation unit 219, and the temperature calculation unit 220 (steps S403 to S415 in FIG. 1) are similar to those in steps S103 to S115. Setting different from steps S103 to S115 are as described in step S400.

The second control unit 223 executes the processes in steps S401 to S415 for each time Δt until a certain calculation time t=t3 is reached from t=o (YES in step S416 in FIG. 1). The calculation time t3 may be set to a value at which the predicted value T[t+Δt][° C.] of the deep body temperature reaches a steady state.

In the preliminary calculation III, the heat transfer/heat radiation amount calculation unit 215 may use Equations 18 and 19 in a case where the first measurement unit 211 measures the air temperature Tₐ[t][° C.] outside the clothing of the measurement target person, similarly to the main calculation.

### Main Calculation.

In the main calculation, the processes in steps S101 to S115 are performed by using the final values of the temperatures T_{US}[t+Δt], T_{UC}[t+Δt], T_{LS}[t+Δt], T_{LC}[t+Δt], Tₛₖ[t+Δt], and T[t+Δt] calculated through the preliminary calculation III as initial values of the temperatures of each site/each layer.

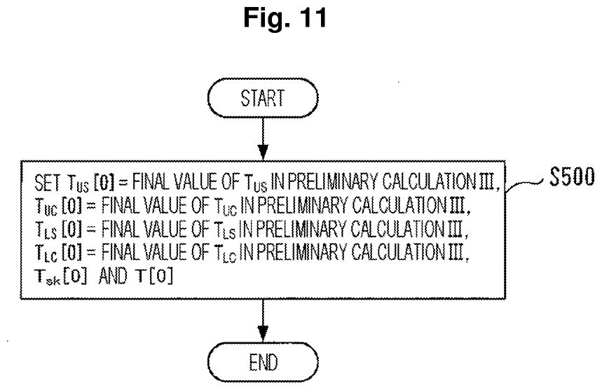

FIG. 11 is a flowchart for describing an operation of the third control unit 224 during the main calculation. Prior to the main calculation, the third control unit 224 sets the final value of T_{US}[t+Δt] in the preliminary calculation III as the initial value T_{US}[O] of the temperature of the trunk skin layer US of the measurement target person, the final value of T_{UC}[t+Δt] in the preliminary calculation III as the initial value T_{UC}[O] of the temperature of the trunk deep layer UC, the final value of T_{LS}[t+Δt] in the preliminary calculation III as the initial value T_{LS}[O] of the temperature of the limb skin layer LS, and the final value of T_{LC}[t+Δt] in the preliminary calculation III as the initial value T_{LC}[O] of the temperature of the limb deep layer LC (step S500 in FIG. 11).

The third control unit 224 sets the values calculated by setting t+Δt=o in Equations 9 and 10 as the initial value Tₛₖ[O] of the average skin temperature and the initial value T[O] of the deep body temperature (step S500 in FIG. 11). The reference temperatures Tₛₖₒ and T₀ remain at the values set in step S400.

The third control unit 224 starts the processes in steps S101 to S115.

As described above, in the present embodiment, not only the information stored in advance in the calculation parameter acquisition unit 1 and the temperature in the vicinity of the measurement target person but also the humidity in the vicinity of the measurement target person is measured, a heat quantity flowing into and out of each site/each layer of the measurement target person is calculated, and the deep body temperature of the measurement target person is predicted from the calculated heat quantity. Therefore, the deep body temperature can be predicted with higher accuracy than in the second embodiment. In the present embodiment, an initial value of the temperature of each site/each layer can be set by performing preliminary calculation prior to main calculation.

The skin transpiration amount calculation unit 216 may be used as the water loss amount prediction unit 21 described in the first embodiment. That is, the sum E [W] of insensitive transpiration and sensitive transpiration calculated by the skin transpiration amount calculation unit 216 is a water loss amount during work of the measurement target person.

In the present embodiment, a temperature in the vicinity of the measurement target person is measured by the first measurement unit 211, and a humidity in the vicinity of the measurement target person is measured by the second measurement unit 212. However, a predicted value of the temperature Tₐ[t][° C.] in the vicinity of the measurement target person on the day of work of the measurement target person and a predicted value of the relative humidity humidity[t][%] in the vicinity of the measurement target person may be acquired from the calculation parameter acquisition unit 1 and used.

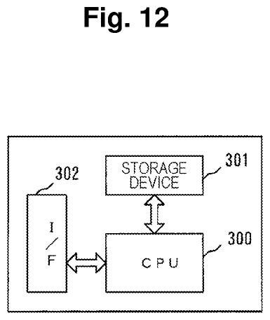

The calculation parameter acquisition unit 1, the biological information prediction unit 2, the time prediction unit 3, and the heat risk notification unit 4 of the first to third embodiments can be realized by a computer including a central processing unit (CPU), a storage device, and an interface, and a program for controlling these hardware resources. A configuration example of this computer is illustrated in FIG. 12.

The computer includes a CPU 300, a storage device 301, and an interface device (I/F) 302. Hardware of a circuit unit of the calculation parameter acquisition unit 1, hardware of a circuit unit of the biological information prediction unit 2, hardware of a circuit unit of the heat risk notification unit 4, and the like are connected to the I/F 302. In such a computer, a heat risk notification program for realizing the notification method of the present embodiment is stored in the storage device 301. The CPU 300 executes the processes described in the first to third embodiments according to the program stored in the storage device 301. The program may also be provided via a network.

### INDUSTRIAL APPLICABILITY

Embodiments of the present invention can be applied to a technique for preventing heat stroke.

### REFERENCE SIGNS LIST

1 Calculation parameter acquisition unit
2 Biological information prediction unit
3 Time prediction unit
4 Heat risk notification unit
20 Deep body temperature prediction unit
21 Water loss amount prediction unit
201 Measurement unit
202 First calculation unit
203 Second calculation unit
211 First measurement unit
212 Second measurement unit
213 Calorific value calculation unit
214 Metabolic rate calculation unit
215 Heat transfer/heat radiation amount calculation unit
216 Skin transpiration amount calculation unit
217 Exhalation transpiration amount setting unit
218 Heat exchange amount calculation unit
219 Heat exchange amount calculation unit
220 Temperature calculation unit
221 Heat quantity calculation unit
222 First control unit
223 Second control unit
224 Third control unit

1.-8. (canceled)

9. A notification device comprising:
a calculation parameter acquisition circuit configured to acquire information regarding physical features, work conditions, and environmental conditions of a measurement target person;
a biological information prediction circuit configured to predict biological information of the measurement target person for each of a plurality of times in which the measurement target person performs work under conditions specified by the work conditions and the environmental conditions based on the information acquired by the calculation parameter acquisition circuit;
a time prediction circuit configured to predict a time at which the biological information predicted by the biological information prediction circuit will exceed a predetermined threshold value; and
a heat risk notification circuit configured to provide a notification of heat countermeasure information based on the time predicted by the time prediction circuit.

10. The notification device according to claim 9, wherein:
the information regarding the physical features comprises information regarding a height, a weight, an age, and a sex of the measurement target person;
the information regarding the work conditions comprises information regarding clothing and a metabolic equivalents value of the measurement target person; and
the information regarding the environmental conditions comprises information regarding a temperature and a humidity on a day of and at a place where the measurement target person is performing the work.

11. The notification device according to claim 10, wherein:
the biological information comprises a deep body temperature of the measurement target person;
the time prediction circuit is configured to predict a time at which the deep body temperature predicted by the biological information prediction circuit will exceed a predetermined deep body temperature threshold value; and
the heat countermeasure information comprises information for reporting the time predicted by the time prediction circuit as a time at which the measurement target person will be exposed to an excessive heat load.

12. The notification device according to claim 10, wherein:
the biological information comprises a water loss amount of the measurement target person;
the time prediction circuit is configured to predict a time at which the water loss amount predicted by the biological information prediction circuit will exceed a predetermined water loss amount threshold value; and
the heat countermeasure information comprises information for reporting the time predicted by the time prediction circuit as a time at which the measurement target person will be exposed to an excessive heat load.

13. The notification device according to claim 10, wherein:
the biological information comprises a deep body temperature and a water loss amount of the measurement target person;
the time prediction circuit is configured to predict a time at which the deep body temperature predicted by the biological information prediction circuit will exceed a predetermined deep body temperature threshold value and a time at which the water loss amount predicted by the biological information prediction circuit will exceed a predetermined water loss amount threshold value; and
the heat countermeasure information comprises information for reporting an earlier one of the two times predicted by the time prediction circuit as a time at which the measurement target person will be exposed to an excessive heat load.

14. The notification device according to claim 13, wherein the heat countermeasure information comprises the biological information predicted by the biological information prediction circuit in addition to the information regarding the time at which the measurement target person will be exposed to the excessive heat load.

15. The notification device according to claim 9, wherein:
the biological information comprises a deep body temperature of the measurement target person;
the time prediction circuit is configured to predict a time at which the deep body temperature predicted by the biological information prediction circuit will exceed a predetermined deep body temperature threshold value; and
the heat countermeasure information comprises information for reporting the time predicted by the time prediction circuit as a time at which the measurement target person will be exposed to an excessive heat load.

16. The notification device according to claim 9, wherein:
the biological information comprises a water loss amount of the measurement target person;
the time prediction circuit is configured to predict a time at which the water loss amount predicted by the biological information prediction circuit will exceed a predetermined water loss amount threshold value; and
the heat countermeasure information comprises information for reporting the time predicted by the time prediction circuit as a time at which the measurement target person will be exposed to an excessive heat load.

17. The notification device according to claim 9, wherein:
the biological information comprises a deep body temperature and a water loss amount of the measurement target person;
the time prediction circuit is configured to predict a time at which the deep body temperature predicted by the biological information prediction circuit will exceed a predetermined deep body temperature threshold value and a time at which the water loss amount predicted by the biological information prediction circuit will exceed a predetermined water loss amount threshold value; and
the heat countermeasure information comprises information for reporting an earlier one of the two times predicted by the time prediction circuit as a time at which the measurement target person will be exposed to an excessive heat load.

18. The notification device according to claim 17, wherein the heat countermeasure information comprises the biological information predicted by the biological information prediction circuit in addition to the information regarding the time at which the measurement target person will be exposed to the excessive heat load.

19. A notification method comprising:
acquiring information regarding physical features, work conditions, and environmental conditions of a measurement target person;
predicting biological information of the measurement target person for each of a plurality of times in which the measurement target person performs work under conditions specified by the work conditions and the environmental conditions based on the acquired information;
predicting a time at which the predicted biological information will exceed a predetermined threshold value; and
providing notification of heat countermeasure information based on the predicted time.

20. The notification method according to claim 19, wherein:
the information regarding the physical features comprises information regarding a height, a weight, an age, and a sex of the measurement target person;
the information regarding the work conditions comprises information regarding clothing and a metabolic equivalents value of the measurement target person; and
the information regarding the environmental conditions comprises information regarding a temperature and a humidity on a day of and at a place where the measurement target person is performing the work.

21. The notification method according to claim 19, wherein:
the biological information comprises a deep body temperature of the measurement target person;
predicting the time at which the predicted biological information will exceed the predetermined threshold value comprises predicting a time at which the deep body temperature of the measurement target person will exceed a predetermined deep body temperature threshold value; and
the heat countermeasure information comprises information for reporting the predicted time as a time at which the measurement target person will be exposed to an excessive heat load.

22. The notification method according to claim 19, wherein:
the biological information comprises a water loss amount of the measurement target person;
predicting the time at which the predicted biological information will exceed the predetermined threshold value comprises predicting a time at which the predicted water loss amount will exceed a predetermined water loss amount threshold value; and
the heat countermeasure information comprises information for reporting the predicted time as a time at which the measurement target person will be exposed to an excessive heat load.

23. The notification method according to claim 19, wherein:
the biological information comprises a deep body temperature and a water loss amount of the measurement target person;
predicting the time at which the predicted biological information will exceed the predetermined threshold value comprises predicting a time at which the predicted deep body temperature will exceed a predetermined deep body temperature threshold value and a time at which the predicted water loss amount will exceed a predetermined water loss amount threshold value; and
the heat countermeasure information comprises information for reporting an earlier one of the two predicted times as a time at which the measurement target person will be exposed to an excessive heat load.

24. The notification method according to claim 23, wherein the heat countermeasure information comprises the predicted biological information in addition to the information regarding the time at which the measurement target person will be exposed to the excessive heat load.

25. A non-transitory computer-readable storage medium for storing a program that when executed by a computer causes a notification device to perform the method according to claim 19.
